(19) United States
(12) Patent Application Publication
(10) Pub. No.: US 2007/0027366 A1
(43) Pub. Date: Feb. 1, 2007

(54) Device and system for entering and monitoring dietary data

(76) Inventor: Jonathan W. Osburn, Union Mills, IN (US)

Correspondence Address: HARSHAW RESEARCH INCORPORATED, P O BOX 418, OTTAWA, KS, 66067, US

(21) Appl. No.: 11/191,567

(22) Filed: Jul. 28, 2005

(51) Int. Cl. A61B 5/00

(52) U.S. Cl. 600/300; 128/921

(57) ABSTRACT

A system for entering and monitoring dietary data for use with a computer includes a handheld electronic device having an input mechanism for inputting dietary data. The handheld device may be interfaced with the computer for communication therebetween. The system includes software for operation upon the computer, which includes instructions for retrieving the dietary information from the electronic device. The computer includes a printer and display and the software includes instructions enabling the dietary information to be analyzed and for the analyzed data to be displayed graphically on the display. Nutritional data may also be printed as a barcode for application to a label or restaurant menu. Dietary data, in the form of recipes or recipe libraries, may be input directly into the computer and then uploaded to the electronic device for user selection. The system provides storage, analysis, and comparisons of actual consumption and target consumption data.

### BACKGROUND OF THE INVENTION

This invention relates generally to a monitoring device and system. In particular, the present invention relates to a device and system for monitoring dietary data such as quantities and types of foods, ingredients, or nutritional elements consumed in a relative period of time.

Improvement in one's diet requires accurate and well-kept records. Unfortunately, recording three meals a day worth of nutritional information is difficult and an unrealistic expectation for most people. Various proposals for devices that monitor dietary data are known in the art, such as in U.S. Pat. Nos. 6,675,041, 5,890,128, 5,704,350, 4,380,802 and Application 2002/0,027,164. Although assumably effective for their intended purposes, these systems do not provide an efficient solution to recording one's nutritional intake at remote locations throughout the day for later interfacing with a personal computer having specialty software for analyzing the dietary data and selectively communicating said analyzed data to a remote third party over a computer network.

Therefore, it is desirable to have an efficient and accurate method and system for entering and monitoring dietary information. Further, it is desirable to have a system that enables a user to enter dietary data into a handheld electronic device that may be later interfaced to the user's personal computer. Still further, it is desirable to have a system that includes computer software capable of analyzing dietary data, displaying or printing appropriate charts and graphs, and selectively communicating data to remote locations over the Internet, e.g. to a doctor's office. It is also desirable that analysis of entered dietary data can be related to a time component.

### SUMMARY OF THE INVENTION

Accordingly, a system for entering and monitoring dietary information includes a handheld electronic device having an input mechanism for enabling a user to input dietary information, such as ingredients in a recipe or individual ingredients in a food item eaten at a restaurant. The system also includes a software product having a plurality of program instructions for execution by a computer. The handheld electronic device may be interfaced with the computer with appropriate wiring or wireless connections. The software program instructions permit the software to retrieve dietary information previously input into the electronic device, e.g. food items or ingredients consumed and to estimate nutritional data therefrom. Thus, a user may enter data concerning consumption of food items over a time period and the program instructions are capable of analyzing the dietary information or estimating its nutritional value. The program instructions, when executed, enable the data to be displayed in text or graphical form on the computer's display.

Similarly, recipe data may be input directly into the computer and the program instructions enable such data to be uploaded into the electronic device. For example, recipes or dietary information relative to a food category, e.g. Mexican food, may be entered into the computer under software control and then interfaced to the handheld electronic device. Having the “Mexican food” library of data in the handheld device enables much easier and faster entry of relevant consumed food items.

Therefore, a general object of this invention is to provide a system for entering and monitoring dietary information including a handheld electronic device with data entry mechanisms.

Another object of this invention is to provide a system, as aforesaid, in which the handheld electronic device may interface with a personal computer.

Still another object of this invention is to provide a system, as aforesaid, including a software product for use with a computer, the software product having program instructions for receiving data from the interfaced electronic device.

Yet another object of this invention is to provide a system, as aforesaid, which includes a recipe-driven user interface for quick and easy entry of foods being consumed.

A further object of this invention is to provide a system, as aforesaid, in which dietary data can be communicated to health care professionals for further analysis and recommendation.

A still further object of this invention is to provide a system, as aforesaid, which allows new recipes or recipe libraries to be entered into the computer via the software product and then to be uploaded to the handheld electronic device for aiding in later dietary data entry.

A particular object of this invention is to provide a system, as aforesaid, in which entered ingredients or food selected from a recipe library may be analyzed for final product nutritional properties.

Another object of this invention is to provide a system, as aforesaid, in which a barcode associated with particular recipe data may be generated and printed.

Still another object of this invention is to provide a system, as aforesaid, in which personal health data such as cholesterol, blood sugar, and lipid levels and blood pressure may be entered, stored, and analyzed.

Yet another object of this invention is to provide a system, as aforesaid, in which actual consumption statistics may be tracked over time and compared with target consumptions goals.

Other objects and advantages of this invention will become apparent from the following description taken in connection with the accompanying drawings, wherein is set forth by way illustration and example, an embodiment of this invention.

### BRIEF DESCRIPTION OF THE DRAWINGS

FIG. 1 is a perspective view of a handheld electronic device according to a preferred embodiment of a system for entering and monitoring dietary information;

FIG. 2 is a block diagram of the system for entering and monitoring dietary information;

FIG. 3 is a schematic diagram indicating the functionality and logic of the system of FIG. 2; and

FIG. 4 is a schematic diagram showing the function and logic of the system of FIG. 2.

### DESCRIPTION OF THE PREFERRED EMBODIMENT

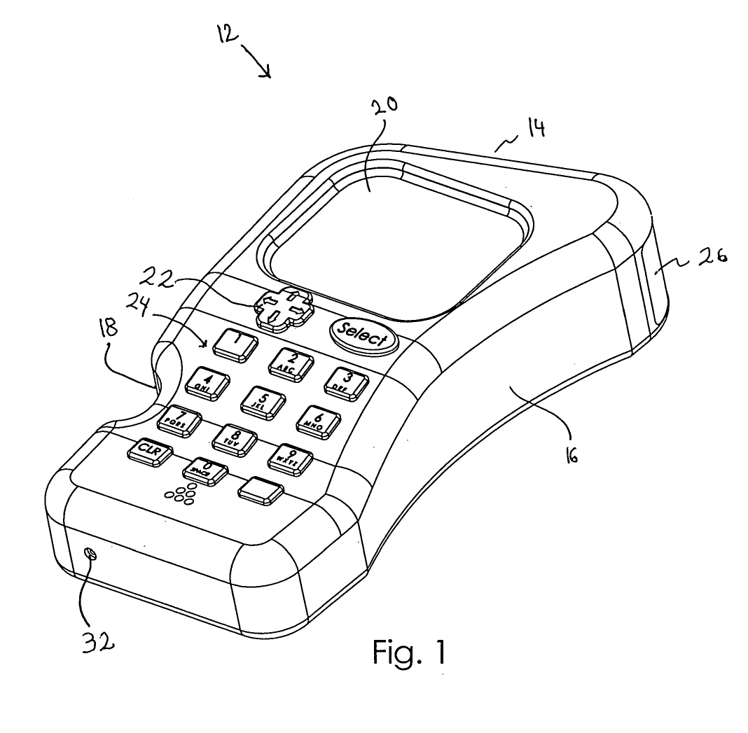

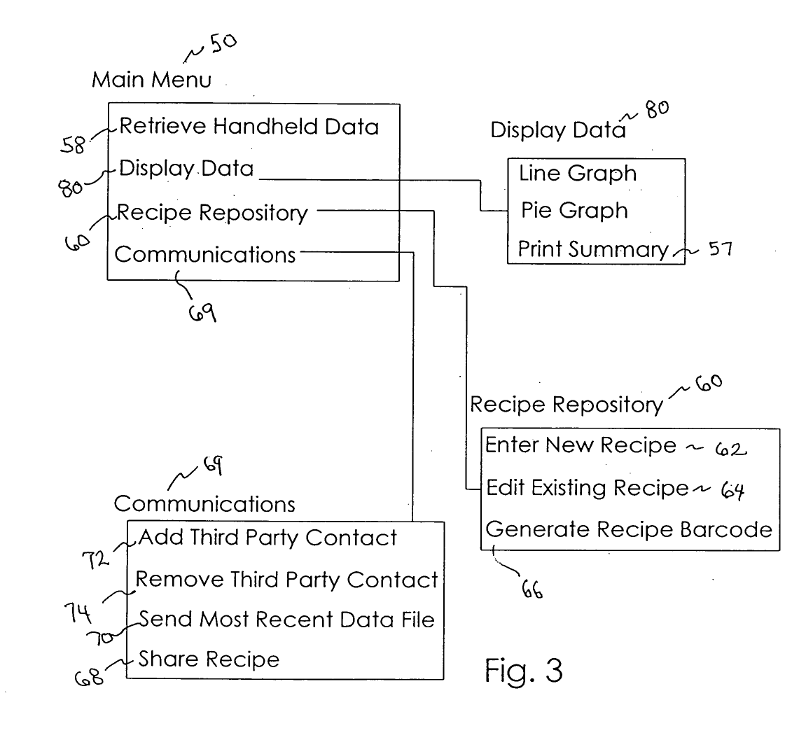

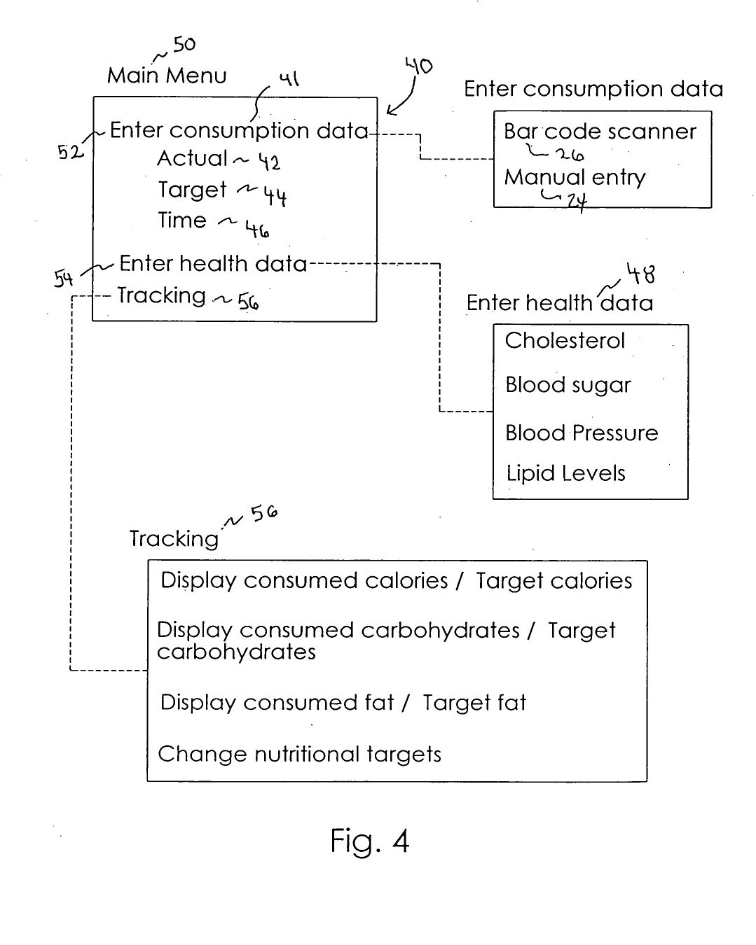

A device and system for monitoring dietary data according to the present invention will now be described in detail with reference to FIGS. 1 through 4 of the accompanying drawings. The system according to the present invention includes a handheld device 12 (FIG. 1) and a software product (FIGS. 3 and 4) for operation upon a personal/desktop computer 8 having a traditional setup of an input device(s), display, and printer (not shown). The software product includes instructions stored on a computer readable medium for operation upon the computer 8.

The handheld device 12 includes a housing 14 having an ergonomic configuration (FIG. 1). Specifically, the housing 14 includes a generally arcuate shape on one side wall 16 and a notch on an opposed side wall 18 for enhanced grip by a user's fingers and thumb, respectively. The handheld device 12 further includes a display screen 20, navigation buttons 22, and a keypad 24 for data entry. A barcode scanner 26 is also provided in the handheld device 12 for reading dietary information from special barcodes indicative of dietary information. Further, the handheld device 12 includes a memory 28 and a central processing unit 30 for storage and processing of data, respectively, as will be described in more detail below. A data port 32 is positioned in a bottom wall of the housing for interfacing the handheld device 12 with the personal computer 8 for electronic communication therebetween. The software product for the personal computer 8 is a companion to the handheld device 12 for organizing dietary, health, and time data items as will be explained herein. Data may be entered either at the handheld device 12 or at the personal computer 8 and may be transferred therebetween for a user's convenience.

In general, the handheld device 12 will be utilized by a user to record consumption of foods at the time and location of consumption. In other words, a user may enter what foods are consumed at a restaurant, say, during lunch. Each of these food items is associated with predetermined nutritional values and, over time, nutritional patterns may be established through analysis of the entered data.

Data must be well organized in order to accomplish this invention with efficiency. Accordingly, the present system includes unique data structures to accomplish the many objects of the invention. As best shown in FIG. 4, the system includes dietary records 40 that may be stored electronically in memory locations on the desktop computer 8 and in the memory 28 of the handheld device 12. Each record 40 includes a plurality of data fields for storing consumption data 41 relative to actual consumption 42, consumption targets 44, time data 46, and other health data. More particularly, the actual 42 and target consumption 44 data may be further defined regarding calories, carbohydrates, fat, sodium, cholesterol, or other nutritional or chemical components. As shown at 48, the general health data fields are associated with data concerning a user's cholesterol, blood sugar, blood pressure, lipid levels, or the like. The software product includes instructions to present a Main Menu 50 enabling a user to enter consumption data 52, enter health data 54, or track previously entered data over time 56 (FIG. 4). Of course, this data is preferably entered into the handheld device 12 and later downloaded/interfaced to the computer software. It is understood that the handheld device 12 includes similar software for collecting this data in a manner substantially similar as that of the computer software, as described below.

Actual consumption data 42 is input into the handheld device 12, and later downloaded back to the personal computer 8, stored in the data structures described above, and analyzed. More particularly, actual consumption data 42 is entered into the handheld device 12 and stored in respective memory locations. It should be understood that many of the data structures described above are duplicated in the handheld device 12 as they may be uploaded and downloaded between the computer 8 and handheld device 12. Actual consumption data 42 may be entered using the keypad 24 or barcode scanner 26 (FIG. 1). To aid in this process, a recipe repository 60 may be stored in the handheld device's memory 28. For example, a Mexican food recipe family may be uploaded from the desktop computer 8 to the handheld device 12. Then, a user may quickly select from the recipe repository 60 what foods or ingredients were actually consumed. This information can later be downloaded to the software product on the desktop computer 8 for analysis as will be described in more detail later. Restaurants that support and have implemented the system disclosed herein may have affixed barcodes on their menus which correspond to the dietary information included in a recipe family or that the user would otherwise enter manually into the handheld device 12. In that case, the user may simply scan the barcode with the barcode scanner 26 and the dietary information is captured for later tracking and analysis. It is understood that the handheld device 12 also includes software having instructions stored on computer readable media and executed on a handheld device CPU for the entry, uploading, downloading, and processing of data.

It should be appreciated that the software product includes instructions that enable a user to enter a new recipe 62, edit an existing recipe 64, and generate a recipe barcode 66 (FIG. 3). A generated barcode may be printed out and affixed to a menu by a restaurant owner or used by the user to speed data entry in the future for frequently consumed ingredients. Entered or edited recipes, of course, may be uploaded to the handheld device 12 and stored in memory 28 for later use in entering consumed ingredients.

Entry of general health information is managed in a substantially similar manner. In other words, data such as cholesterol, blood sugar, blood pressure, or lipid levels may be entered into the handheld device 12 using the keypad 24 and later interfaced to the desktop computer 8 and software, or entered directly into the software at the desktop computer 8 (FIG. 4) for analysis.

The software product includes instructions for analyzing and tracking a user's dietary information. When a user inputs dietary consumption data, the software associates this data with its database of ingredients for the purpose of estimating the nutritional value thereof. For example, each ingredient in a recipe in the recipe repository 60 is associated with a predetermined quantity of carbohydrates, fat, cholesterol or the like. This predetermined association provides the basis for estimated dietary data. The software product makes these estimations when consumption data is downloaded from the handheld device 12 to the desktop computer 8. The software may then selectively direct the dietary data to the computer's printer 57 as text or in a computer readable format such as a barcode (FIG. 3). Printing the estimated dietary data as a barcode enables this information to be entered much faster and easier the next time a particular ingredient is consumed by using the barcode scanner 26.

Once both consumption and general health information has been entered into the handheld device 12 and downloaded to the desktop computer 8 or directly entered into the computer 8, this data may be analyzed as a function of time and relative to particular ingredients or nutritional elements. For example, the software can track a user's fat intake over a period of weeks or months. Then, the analyzed data may be displayed 80 on the computer display in the form of text, line graphs, pie charts, and the like (FIG. 3). Of course, it may be printed 57 as well.

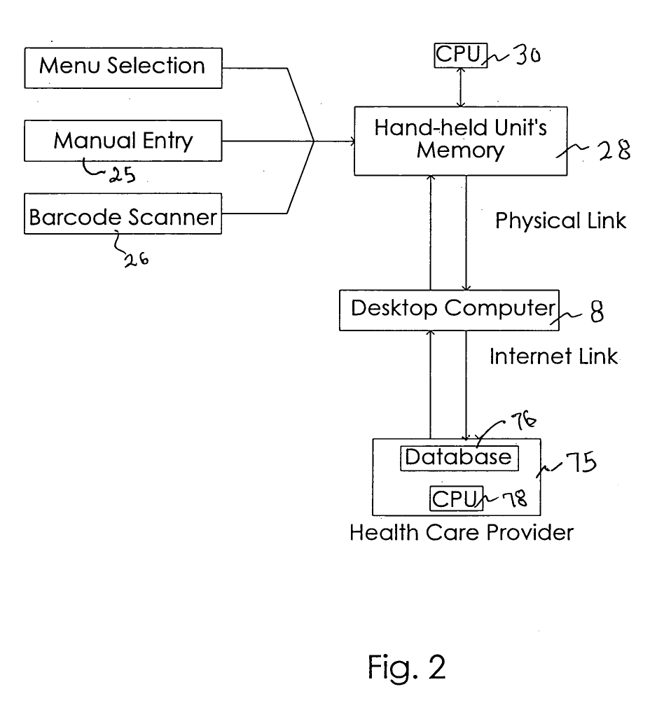

Another important aspect of this invention is to communicate a user's dietary information to remote third parties using the Internet so that the user may receive important feedback. More particularly, the software include a “Communication” menu selection 69 enabling a user to add 72 or remove 74 a third party contact 75, to send a most recent dietary data file 70, or to communicate recipes or a recipe library with a third party contact 68 (FIG. 3). This is a unique aspect of the system in that actual consumption data has never before been immediately communicable to health or nutritional advisors. Third party advisors may then return comments or advice to the user. It is understood that a third party contact may be the user's health care provider who, being a participant in the present system, may correlate transmitted data with the user's health records stored in a database 76 (FIG. 2). With software in operation upon a third party CPU 78, analysis of the transmitted data may be performed so as to make recommendations back to the user.

In use, recipe data or recipe libraries may be uploaded from the desktop computer 8 to the handheld device and stored in memory 28 thereon (FIG. 2). The handheld device 12 may be organized to include a database of uploaded recipe data (i.e. a recipe repository 60) wherein each ingredient in the recipe or library is associated with an estimated nutritional value. Recipe data may be entered 62, edited 64, or even shared 68 via a computer network from the desktop computer (FIG. 3). Then, dietary data may be input into the handheld device 12 from the location and at the time of consumption using the keypad 24 and other input keys (manual entry 25) or with the barcode scanner 26. A time aspect is recorded in a respective dietary record at the time it is entered. Entered data and estimated dietary data may be selectively retrieved/downloaded 58 to the computer 8 to be processed according to the companion software (FIG. 3). Selected aspects of consumed ingredients may be analyzed relative to the time aspect and displayed 80 on the computer display. Analyzed or raw data may also be selectively communicated to third parties for further analysis or comment 70 (FIG. 3).

It is understood that while certain forms of this invention have been illustrated and described, it is not limited thereto except insofar as such limitations are included in the following claims and allowable functional equivalents thereof.

What is claimed is as follows:

1. A system for entering and monitoring dietary data for use with a computer having a display, an input device, and a printer, said system comprising:
a handheld electronic device having an input mechanism for inputting dietary data;
means for interfacing said handheld device with the computer for communication between said handheld device and the computer; and
a software product comprising instructions stored on computer-readable media, wherein said instructions, when executed by the computer, perform steps for retrieving data from said handheld device and displaying said data on the computer display.

2. The system as in claim 1, wherein:
said software product includes instructions for accepting recipe data input through the input device of the computer;
said software product includes instructions for estimating dietary data from said recipe data; and
said software product further comprises instructions for transferring said recipe and estimated dietary data to said handheld device

3. The system as in claim 2, wherein said software product further comprises instructions for actuating the printer to print said estimated dietary data in a computer-readable format.

4. The system as in claim 2, wherein said software product includes instructions for analyzing said dietary data and said estimated dietary data.

5. The system as in claim 3, wherein:
said handheld device input mechanism comprises a barcode scanner; and
said computer-readable format is a barcode.

6. The system as in claim 4, wherein said software further comprises instructions for displaying graphical representations on the display indicative of said analyzed dietary data and said estimated dietary data.

7. The system as in claim 6, wherein said software product further comprises instructions for communicating with a remote user over a network.

8. The system as in claim 1, wherein said handheld device includes:
a handheld software product comprising instructions stored on computer-readable media, wherein said instructions, when executed by said handheld device, perform steps for storing dietary records, including dietary consumption data, time data, and personal health data.

9. The system as in claim 8, wherein said handheld device includes:
a database relating said dietary data to predetermined recipe data; and
a user-friendly interface for locating and selecting said recipe data in said handheld device database.

10. The system as in claim 9, wherein:
said dietary data includes a nutritional component and a time component; and
said instructions for displaying said dietary data on the computer display include instructions for displaying line graphs of said dietary data nutritional component versus said dietary data time component.

11. The system as in claim 1, wherein said handheld device input mechanism comprises a keypad.

12. The system as in claim 8, wherein:
said personal health data includes cholesterol, blood sugar, lipid, and blood pressure fields; and
said dietary records include actual consumption and target consumption fields.

13. A dietary data entry and tracking system that utilizes a computer having a display and a printer, said system comprising:
providing a handheld electronic device;
providing a software product, stored on computer-readable media, to be executed by the computer;
inputting recipe data into said software product, said software product including instructions for estimating dietary data from said recipe data and for associating said estimated dietary data with said recipe data;
prompting said software product to estimate said dietary data from said recipe data and to associate said estimated dietary data with said recipe data;
interfacing said handheld electronic device with the computer for communication between said handheld electronic device and said software product;
transferring said associated estimated dietary data and said recipe data to said handheld electronic device;
recording a dietary record on said handheld electronic device;
transferring said dietary record from said handheld electronic device to said software product, said software product further comprising instructions for analyzing said dietary record and displaying said analysis on the computer display; and
prompting said software product to analyze said dietary record and to display said analysis on the computer display.

14. The system as in claim 13, wherein said dietary record includes dietary data and time data.

15. The system as in claim 13, wherein:
the computer is connected to a network for communication with remote users;
said software product includes instructions for communicating with a remote user over the network; and
said system further comprising communicating with a remote user over the network.

16. The system as in claim 13, further comprising printing a printout of said estimated dietary data in a computer-readable format with the printer.

17. The system as in claim 16, wherein:
said computer-readable format is a barcode; and
said handheld device includes a barcode scanner for reading said barcode.

18. The system as in claim 16, further comprising placing said printout on a restaurant menu for simplifying data entry of respective dietary data.

19. A system for entering and monitoring dietary data for use with a computer having a display and a printer, said system comprising:
a handheld electronic device;
means for interfacing said handheld device with the computer for communication between said handheld device and the computer; and
a software product comprising instructions, stored on computer-readable media, wherein said instructions, when executed by the computer, perform steps for transferring data from the computer to said handheld device.

20. The system as in claim 19, wherein said software product further comprises instructions for:
retrieving data from said handheld device;
displaying graphical representations indicative of said retrieved data on the computer display;
estimating dietary data from recipe data entered into said software product;
actuating the printer to print said estimated dietary data in a computer-readable format;
analyzing dietary data and estimated dietary data;
displaying another graphical representation indicative of said analyzed dietary data and estimated dietary data on the display; and
communicating said dietary data with a remote user over a network.
